(19) United States
(12) Patent Application Publication
(10) Pub. No.: US 2024/0315726 A1
(43) Pub. Date: Sep. 26, 2024

(54) ONE-PASS CATHETER TUNNELER

(71) Applicant: Dignity Health, San Francisco, CA (US)

(72) Inventors: Arnau Benet, San Francisco, CA (US); Dakota Graham, San Francisco, CA (US)

(21) Appl. No.: 18/575,777

(22) PCT Filed: Jul. 12, 2022
(86) PCT No.: PCT/US22/36872

§ 371 Date: Dec. 29, 2023

Related U.S. Application Data
(60) Provisional application No. 63/272,062, filed on Oct. 26, 2021.
(60) Provisional application No. 63/220,591, filed on Jul. 12, 2021.

(51) Int. Cl. A61B 17/3205; A61B 17/32; A61M 27/00

(52) U.S. Cl. CPC A61B 17/32053; A61M 27/006; A61B 2017/320056; A61B 2217/005

(57) ABSTRACT

A system for inserting a medical tube within the body using a tunneler device enables formation of a tunnel within the body and further enables concurrent insertion of the medical tube within the body as the tunnel is formed, eliminating extra steps required by current technologies for insertion of the medical tube within the body. The tunneler device provides an attachment element that captures the medical tube and further provides a handle slidably mounted along the tunneler device to aid practitioners when inserting the tunneler device into the body. The tunneler device further provides a bend angle to aid practitioners in forming the tunnel past the clavicle of the body. The tunneler device enables practitioners to place medical tubes within the body while reducing time and friction commonly associated with medical tube placement procedures.

### CROSS-REFERENCE TO RELATED APPLICATIONS

This is a PCT application that claims benefit to U.S. Provisional Patent Application Ser. Nos. 63/220,591 and 63/272,062 respectively filed on 12 Jul. 2021 and 26 Oct. 2021, which are herein incorporated by reference in their entireties.

### FIELD

The present disclosure generally relates to a cranial shunt diversion system, and in particular, to a one-pass tunneler system having a tip that enables a practitioner to place a catheter within the same pass as a tunneler.

### BACKGROUND

Cerebrospinal fluid (CSF) diversion is a common need in neurosurgery. This is a simple concept whereby CSF needs to be absorbed (Diverted) by means other than the arachnoid cells, which typically drain into the sinuses. There are many spaces that are constantly absorbing fluid into the general circulation, including the peritoneum (the most common diversion space targeted), the pleura, and more rarely the atrium or gallbladder. The CSF diversion procedure generally requires the insertion of an intracranial catheter to a space containing CSF, the connection of this intracranial catheter to a shunt valve, and the insertion of the intracranial catheter leading from the valve to the end targeted space for absorption of the diverted CSF.

There are different brands and models of cranial shunts that provide different sizes of cranial catheters, customizable shunt valves and connectors for the catheter. Introducing the catheter requires a method to bring the catheter from the cranial incision to an abdominal or thoracic target (forward passing), although in very limited instances there may be the need for passing the catheter from the end target absorption space towards the cranial incision (reverse passing). Both strategies require the same instrumentation. Passing the catheter requires a tunneler device, which is a “T”-shaped metallic instrument used to create the subcutaneous path between incisions. Current tunneler devices include a plastic cannula sheath that keeps the path created by the tunneler device open after removing the tunneler device with backwards motion through the insertion path. The plastic cannula sheath remains in the path and provides a tunnel for the catheter to be passed through the path. Then, the plastic cannula sheath is removed, and the catheter remains in the path. The distal end of the catheter is introduced into the targeted cavity and tied to the shunt valve at the proximal end. However, the most inefficient step of placing a cranial shunt diversion system is the passing of the catheter through the plastic cannula sheath. There is friction between the plastic cannula sheath and the catheter, which can cause the catheter to fail to reach the distal end of the plastic cannula sheath.

There are alternatives to “patch” the problem of friction, specifically when the catheter fails to reach the distal end of the plastic cannula sheath. These patches include placement of a stitch through the catheter and plastic cannula sheath and pulling the plastic cannula sheath (and the catheter) distally, but this can still fracture the catheter and may require re-tunnelling, which is very traumatic to tissues. Another patch is to use wax to plaster the catheter to the sheath, with high risk of detachment of the catheter mid-way through the path when pulling the plastic cannula sheath, this would as well require re-tunneling, again causing increased trauma. In any way, catheter passing is the most time consuming and toiling part of a shunt and increases surgical time, which is well proven to increase infection risk and postoperative complications.

It is with these observations in mind, among others, that various aspects of the present disclosure were conceived and developed.

### BRIEF DESCRIPTION OF THE DRAWINGS

FIG. 1 is an illustration showing a system for inserting a medical tube into the body by a tunneler device;

FIG. 2 is an illustration showing the system of FIG. 1 inserted between a first incision and a second incision;

FIGS. 3A and 3B are a pair of simplified illustrations showing formation of a tunnel within the body and insertion of the medical tube of FIG. 1 into the tunnel;

FIGS. 4A, 4B and 4C are a series of simplified illustrations showing insertion and advancement of the medical tube through the tunneler device of FIG. 1;

FIGS. 5A and 5B are a pair of illustrations showing a first embodiment of an attachment element of the tunneler device of FIG. 1;

FIGS. 6A and 6B are a pair of illustrations showing coupling of the medical tube with the attachment element of FIGS. 5A and 5B;

FIGS. 7A, 7B and 7C are a series of illustrations showing a second embodiment of an attachment element of the tunneler device of FIG. 1;

FIGS. 8A and 8B are a pair of illustrations showing a third embodiment of an attachment element of the tunneler device of FIG. 1;

FIGS. 9A and 9B are a pair of illustrations showing coupling of the medical tube with the attachment element of FIGS. 8A and 8B;

FIGS. 10A and 10B are a pair of illustrations showing a first open configuration and a second closed configuration of a handle of the tunneler device of FIG. 1;

FIG. 10C is an illustration showing engagement of the handle of FIGS. 10A and 10B with the tunneler device of FIG. 1;

FIG. 10D is an illustration showing an exploded view of the handle of FIGS. 10A and 10B;

FIGS. 11A and 11B are a pair of illustrations showing adjustment of the handle of FIGS. 10A and 10B from a distal position and a proximal position along the tunneler device of FIG. 1;

FIGS. 11C and 11D are a pair of illustrations showing two options for removal of the tunneler device of FIG. 1 from the body following insertion of the tunneler device and medical tube of FIG. 1 into the body;

FIG. 12 is an illustration showing an alternate embodiment of the system of FIG. 1; and

FIGS. 13A, 13B and 13C are a series of process flow diagrams showing a method of inserting a medical tube into a body using the system of FIG. 1.

Corresponding reference characters indicate corresponding elements among the view of the drawings. The headings used in the figures do not limit the scope of the claims.

### DETAILED DESCRIPTION

Various embodiments for a one-pass tunneler device that forms a tunnel within the body and concurrently delivers a medical tube (e.g., a catheter) within the body in one pass are disclosed herein. In a primary embodiment, the tunneler device includes an inner lumen and an attachment element that capture the medical tube therein prior to insertion of the tunneler device within the body, allowing concurrent placement of the medical tube within the tunnel as the tunnel is formed within the body. The tunneler device can then be removed from the body, leaving the catheter within the tunnel. The tunneler device can include a handle to aid practitioners in placement of the tunneler device and catheter within the body. The tunneler device eliminates the extra steps and components required in current catheter placement technologies that involve placing the medical tube within the tunnel following formation of the tunnel, thereby reducing procedure time and friction encountered by the medical tube during placement of the medical tube within the body.

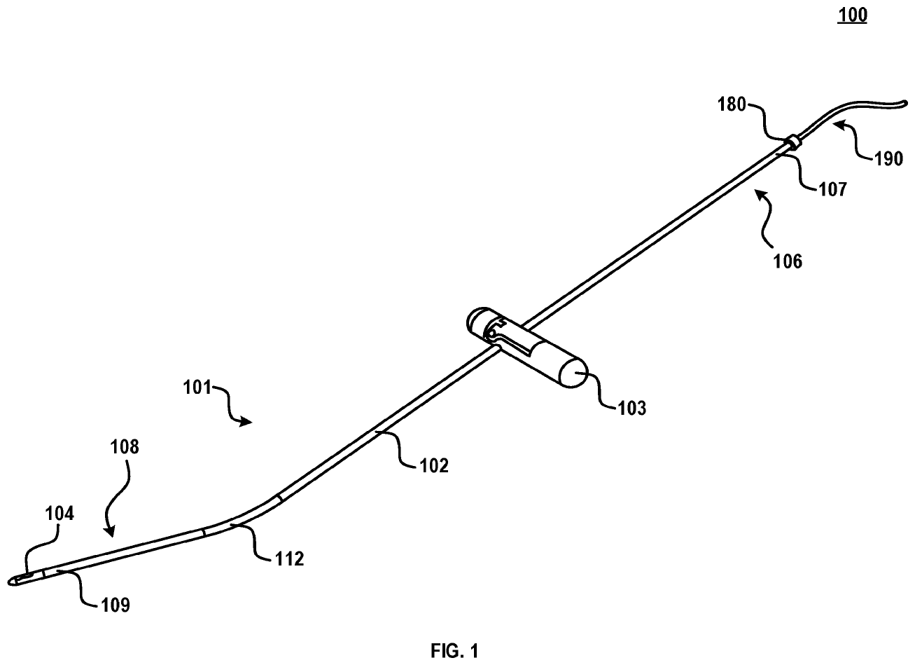

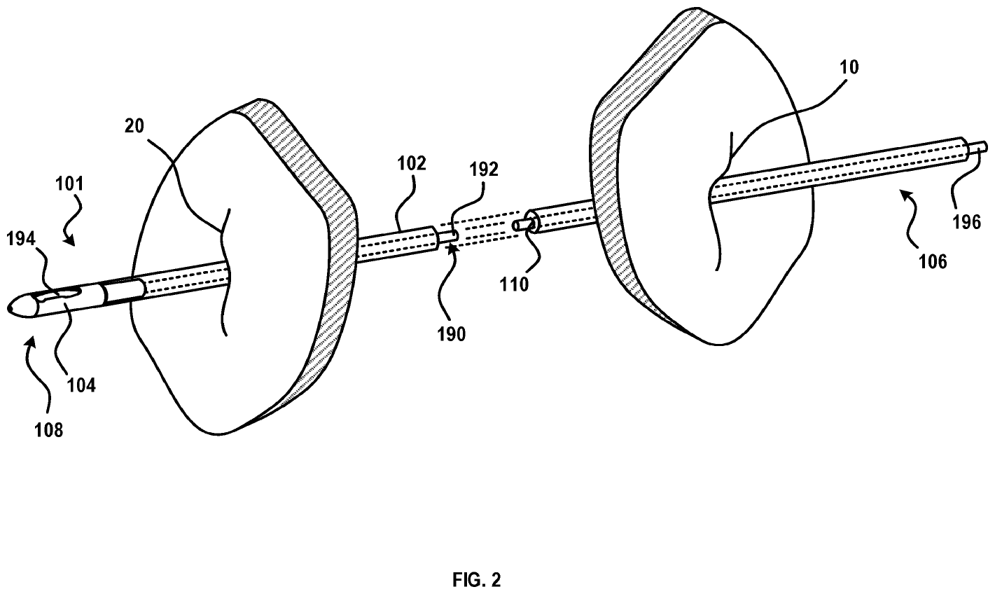

With reference to FIG. 1, a system 100 includes a tunneler device 101 configured to form a tunnel within the body and concurrently place a medical tube 190 within the tunnel. The tunneler device 101 defines a shaft 102 having a proximal portion 106 defining a proximal end 107 and a distal portion 108 defining a distal end 109, where the distal portion 108 is configured for insertion within the body to form a tunnel. In a primary embodiment, the tunneler device 101 configured to receive the medical tube 190 for placement of the medical tube 190 within the tunnel. With additional reference to FIG. 2, the tunneler device 101 includes an inner lumen 110 that receives the medical tube 190 therein prior to insertion within the body to allow concurrent placement of the medical tube 190 within the body as the tunnel is formed. As shown, the tunneler device 101 provides an attachment element 104 at the distal end 109 that secures the medical tube 190 within tunneler device 101 in an arrangement which will be described in greater detail herein. The proximal end 107 can be open and can provide access to the inner lumen 110. The shaft 102 can include a bend angle 112 to aid the practitioner in inserting the tunneler device 101 within the body, particularly past the clavicle. As shown, the tunneler device 101 provides a handle 103 slidably mounted along the shaft 102 of the tunneler device 101 to aid practitioners in inserting the tunneler device 101 within the body. In some embodiments, the tunneler device 101 can include a stopper 180 positioned at the proximal end 107 of the tunneler device 101 that enables connection with a suction generating device to aid practitioners in advancing the medical tube 190 through the tunneler device 101 upon insertion of medical tube 190 into the tunneler device 101. In one aspect, the diameter of the tunneler device 101 may be smaller than standard, conventional tunneler devices because there is no need for a sheath or a second pass of the medical tube, reducing friction and time consumed during placement of the medical tube 190.

A medical professional can manually form the bend angle 112 along the shaft 102 of the tunneler device 101 at their discretion to circumvent a clavicle of the body when tunneling from a cranial incision to an abdominal or thoracic target of a patient. Conversely, the bend angle 112 can be applied along the shaft 102 of the tunneler device 101 during the manufacture of the tunneler device 101. The bend angle 112 can be standardized in order to be utilized by a medical professional when tunneling from a cranial incision to an abdominal or thoracic target of a patient.

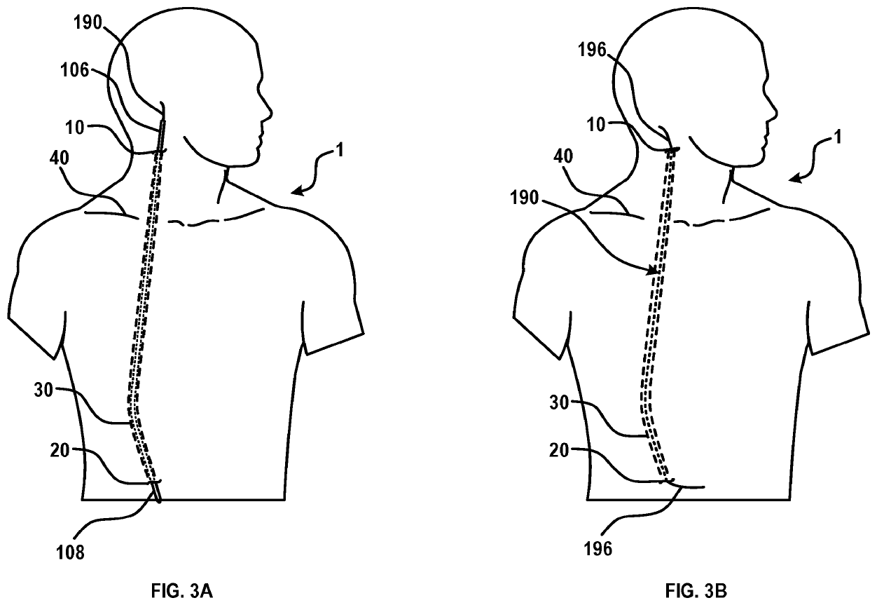

In the examples of FIGS. 2, 3A and 3B, the tunneler device 101 and medical tube 190 are configured for insertion within a body 1 at a first incision 10. The tunneler device 101 and medical tube 190 are further configured for tunneling through the body 1 until the tunneler device 101 and medical tube 190 exit the body at a second incision 20, resulting in formation of a tunnel 30 from the first incision 10 to the second incision 20. In the embodiment shown, the first incision 10 can be a cranial incision positioned superior to a clavicle 40 and the second incision 20 can be at an abdominal or thoracic target of a patient positioned inferior to the clavicle 40. As shown, when fully inserted within the tunnel 30, the proximal portion 106 of the tunneler device 101 extends through the first incision 10 and the distal portion 108 of the tunneler device 101 extends through the second incision 20. The tunneler device 101 can be decoupled from the medical tube 190 and removed from the tunnel 30, leaving the medical tube 190 within the tunnel 30. The tunneler device 101 can be removed either by pulling the tunneler device 101 out of the first incision 10 in the opposite direction of insertion, or by removing the handle 103 and/or stopper 180 and pulling the tunneler device 101 entirely through the tunnel 30 until the proximal end 107 of the tunneler device 101 exits the tunnel 30 at the second incision 20.

The medical tube 190 defines a tube body 192 having a distal portion 194 and a proximal portion 196 defined opposite to the distal portion 194. The tube body 192 can be positioned within the inner lumen 110 of the tunneler device 101 with the proximal portion 196 of the medical tube 190 extending out of the proximal end 107 of the tunneler device 101. The distal portion 194 of the medical tube 190 can couple with the attachment element 104 positioned at the distal end 109 of the tunneler device 101. Insertion of the medical tube 190 into the tunneler device 101 can be completed prior to insertion of the tunneler device 101 into the body and resultant formation of the tunnel 30. As shown, when positioned within the tunnel 30, the proximal portion 196 of the medical tube 190 is positioned at the first incision 10 and the distal portion 194 of the medical tube 190 is positioned at the second incision 20. At this point, the medical tube 190 is ready for attachment to a shunt valve at the proximal portion 196 of the medical tube 190, and for insertion within a target cavity at the distal portion 194 of the medical tube 190.

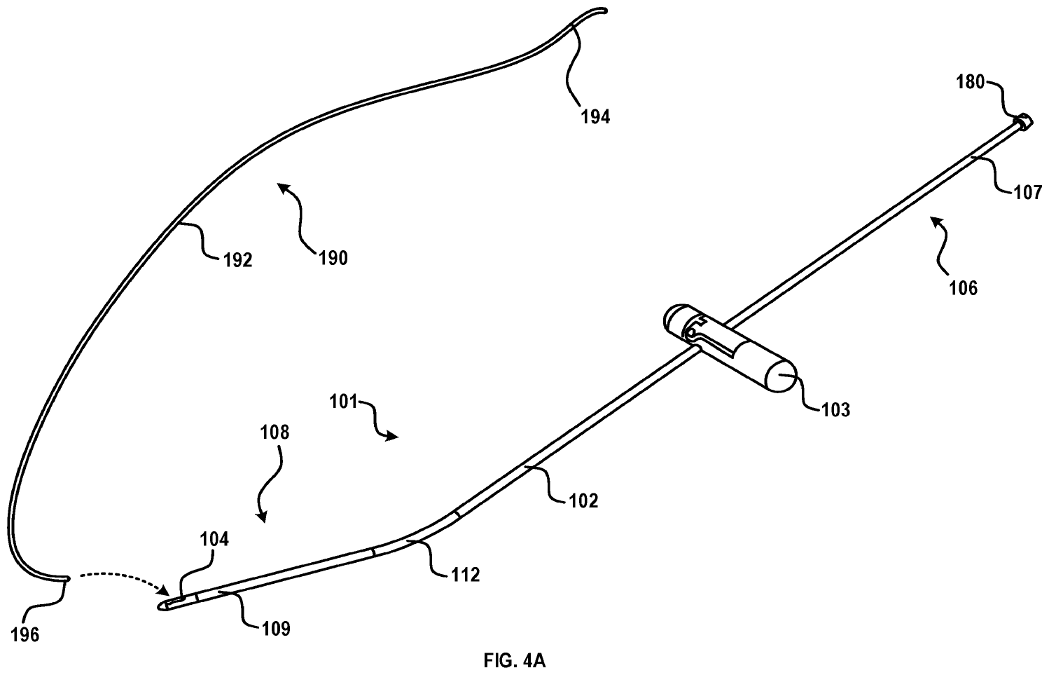

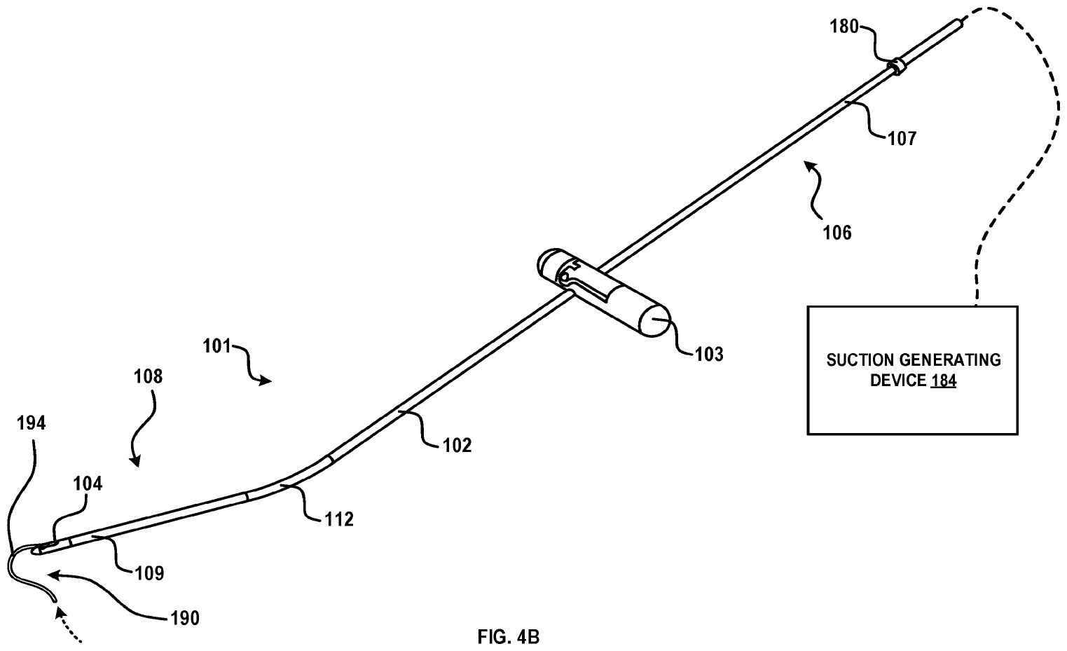

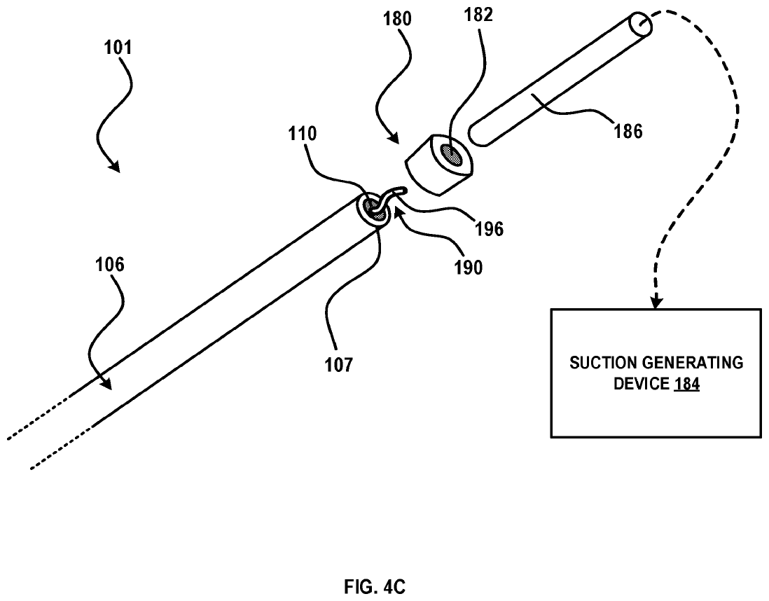

Referring to FIGS. 4A-4C, in some embodiments the medical tube 190 can be inserted into the tunneler device 101 by inserting the proximal portion 196 of the medical tube 190 into the distal end 109 of the tunneler device 101 and advancing the proximal portion 196 of the medical tube 190 through the inner lumen 110 until the proximal portion 196 of the medical tube 190 exits the tunneler device 101 at the proximal end 107 of the tunneler device 101. In a primary embodiment, the medical tube 190 can be inserted into the tunneler device 101 prior Advancement of the medical tube 190 within the inner lumen 110 of the tunneler device 101 can be completed manually or with the aid of the suction generating device 184 as shown in FIG. 4B. In a primary embodiment, the medical tube 190 is inserted within the inner lumen 110 prior to insertion of the tunneler device 101 within the body.

As shown in FIGS. 4B and 4C, the tunneler device 101 provides the stopper 180 that couples the tunneler device 101 to the suction generating device 184. The stopper 180 can include a stopper aperture 182 that enables fluid flow communication between the inner lumen 110 of the tunneler device 101 and the suction generating device 184, and can couple with a suction attachment 186 that connects the suction generating device 184 with the stopper 180. To advance the medical tube 190 through the inner lumen 110, the practitioner can insert the proximal portion 196 of the medical tube 190 into the distal end 109 of the tunneler device 101 and activate the suction generating device 184 to pull the proximal portion 196 of the medical tube 190 through the inner lumen 110 until the proximal portion 196 of the medical tube 190 exits the tunneler device 101 at the proximal end 107 of the tunneler device 101. The stopper 180 can be is attached to the shaft 102 through an anchoring design such as a magnetic, screw-based, and/or friction-fit engagement. In some embodiments, the stopper 180 can include a rubberized and/or flexible material to improve engagement with the suction attachment 186.

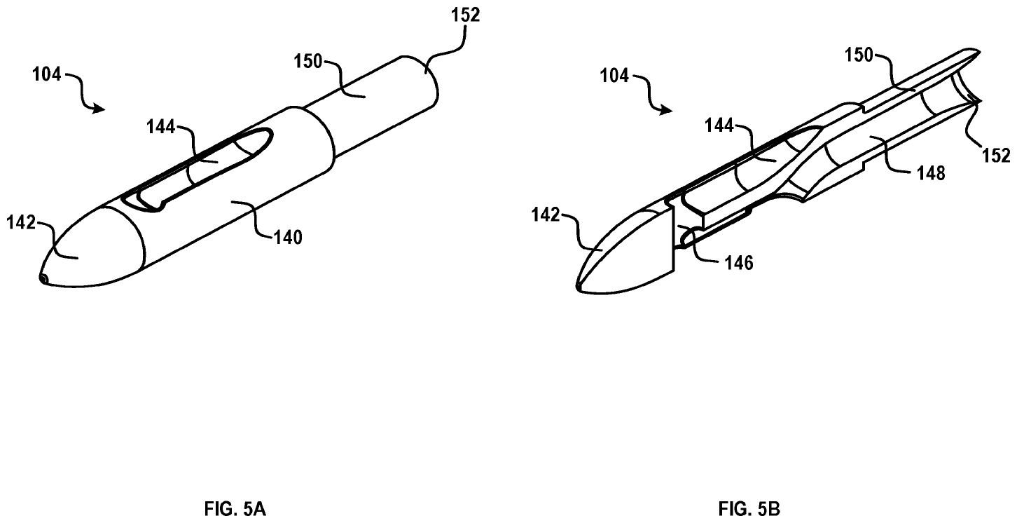

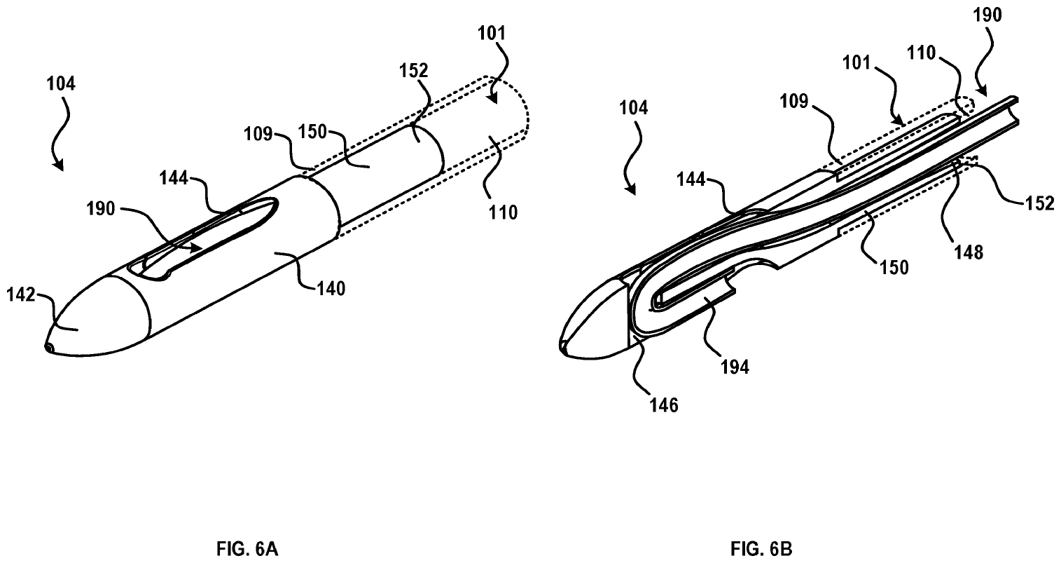

FIGS. 5A-9B show multiple variations of the attachment element 104 (FIGS. 1 and 2) that couples with the distal portion 194 of the medical tube 190. The attachment element 104 occludes the inner lumen 110 at the distal end 109 of the tunneler device 101. As shown in the embodiment of FIGS. 5A and 5B, an attachment element 104 provides an attachment element body 140 defining a tapered distal portion 142 and a cylindrical section 150 defined opposite to the tapered distal portion 142. As shown with additional reference to FIGS. 6A and 6B, a proximal portion 152 of the cylindrical section 150 inserts into the inner lumen 110 at distal end 109 of the tunneler device 101. In some embodiments, the attachment element 104 can be permanently fixed to the shaft 102; in other embodiments, the attachment element 104 can be removably coupled with the shaft 102. The attachment element 104 includes a receptacle 144 that captures the distal portion 194 of the medical tube 190 in a friction-fit engagement. The receptacle 144 forms an entryway for a first channel 146 defined through the attachment element body 140 such that the first channel 146 communicates with an exterior of the tunneler device 101. The first channel 146 communicates with a second channel 148 in coaxial alignment with the inner lumen 110 of the tunneler device 101. The distal portion 194 of the medical tube 190 can be secured in a friction-fit engagement within the first channel 146 and the second channel 148 of the attachment element 104 as shown in FIGS. 6A and 6B.

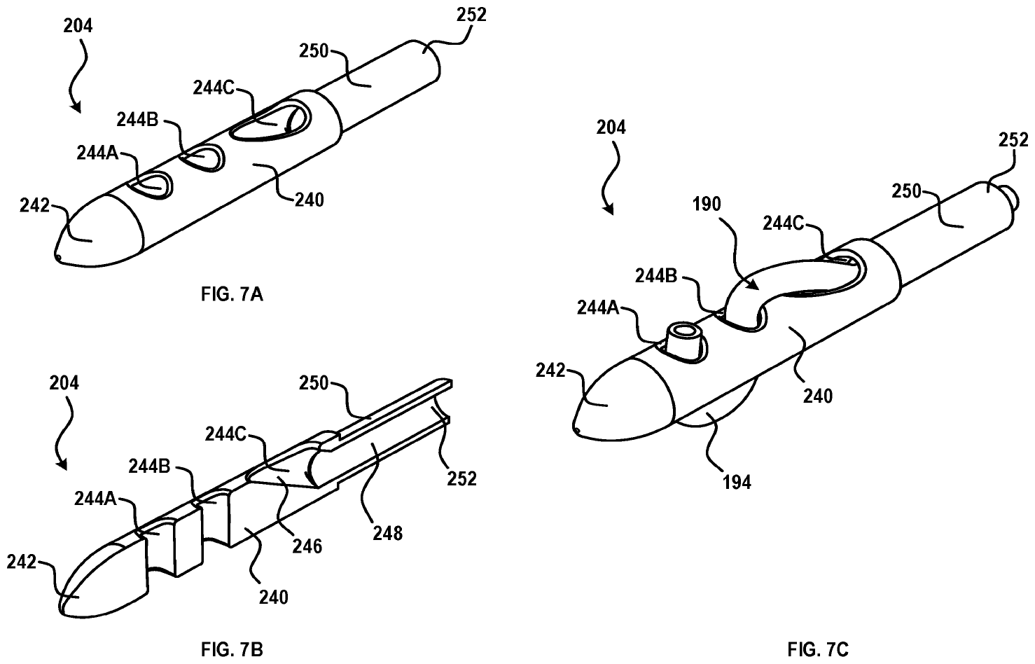

As shown in an alternate embodiment of FIGS. 7A-7C, an attachment element 204 provides an attachment element body 240 defining a tapered distal portion 242 and a cylindrical section 250 defined opposite to the tapered distal portion 242. Similar to the attachment element 104 of FIGS. 5A-6B, a proximal portion 252 of the cylindrical section 250 inserts into the inner lumen 110 at distal end 109 of the tunneler device 101. The attachment element 204 includes a first receptacle 244A, a second receptacle 244B and a third receptacle 244C that all capture the distal portion 194 of the medical tube 190 at three points in a friction-fit engagement. The attachment element body 240 includes a first channel 246 that communicates with an exterior of the tunneler device 101 through the third receptacle 244C. As shown, the first channel 246 communicates with a second channel 248 in coaxial alignment with the inner lumen 110 of the tunneler device 101. The distal portion 194 of the medical tube 190 can be secured in a friction-fit engagement within the first receptacle 244A, the second receptacle 244B, the third receptacle 244C, the first channel 246 and the second channel 248 of the attachment element 204 as shown in FIG. 7C.

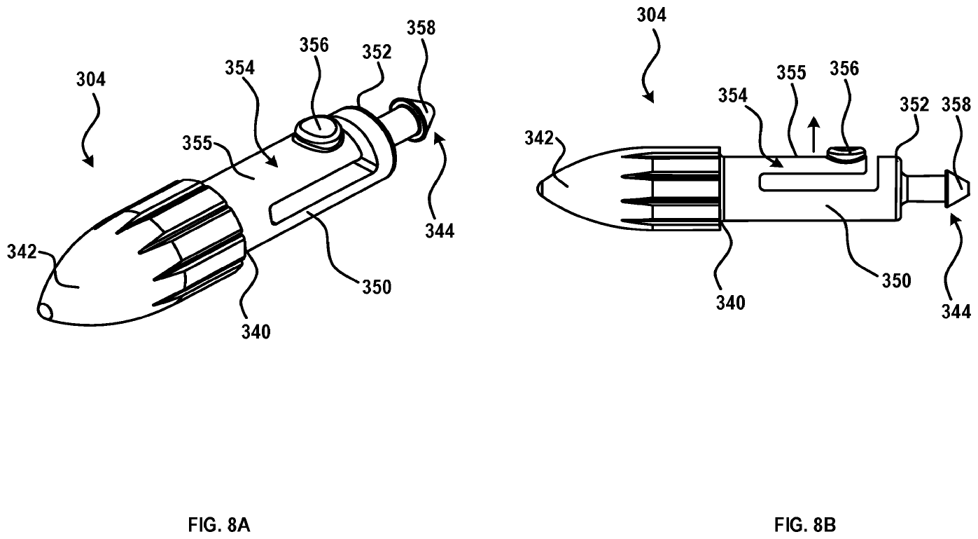

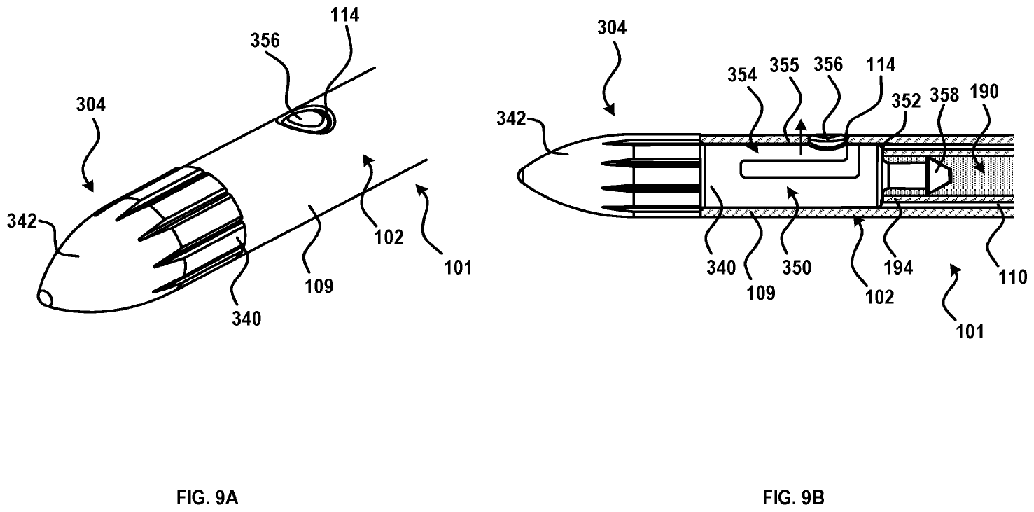

As shown in an alternate embodiment of FIGS. 8A and 8B, an attachment element 304 provides an attachment element body 340 defining a tapered distal portion 342 and a cylindrical section 350 defined opposite to the tapered distal portion 342. Similar to the attachment element 104 of FIGS. 5A-6B and the attachment element 204 of FIGS. 7A-7C, a proximal portion 352 of the cylindrical section 350 inserts into the inner lumen 110 at distal end 109 of the tunneler device 101. The attachment element 304 includes a receptacle 344 in the form of a barb 358 extending from the proximal portion 352 of the cylindrical section 350. With additional reference to FIGS. 9A and 9B, the barb 358 captures the distal portion 194 of the medical tube 190 in a friction-fit engagement. As illustrated, the cylindrical section 350 of the attachment element 304 includes a biased portion 354 having a button 356 along an outer surface 355 of the biased portion 354. The biased portion 354 can be biased in a first radial direction away from a central axis of the attachment element 304. To couple with the attachment element 304, the distal portion 108 of the shaft 102 of the tunneler device 101 includes an aperture 114 configured to receive the button 356 such that actuation of the button 356 in a second radial direction towards the central axis of the attachment element 304 enables decoupling of the attachment element 304 from the distal portion 108 of the shaft 102.

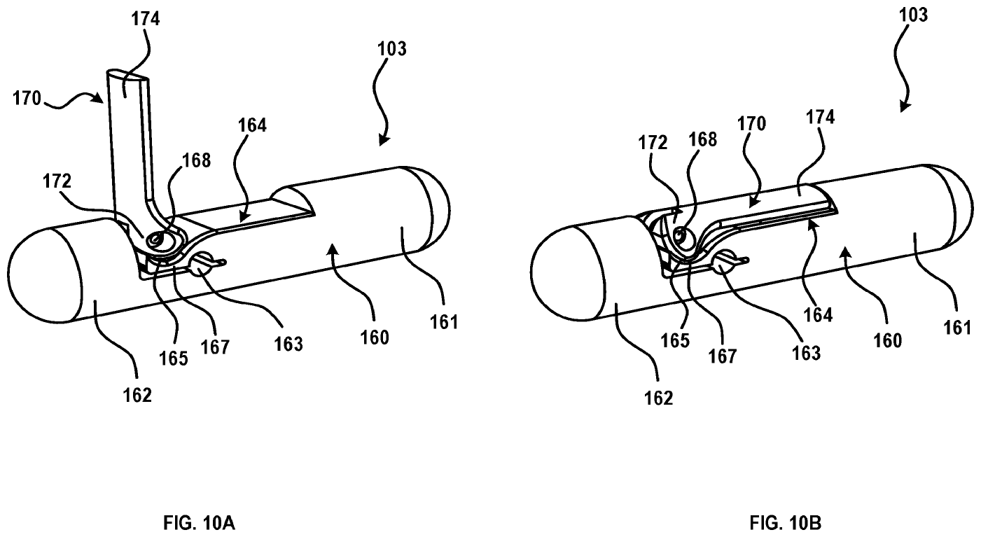

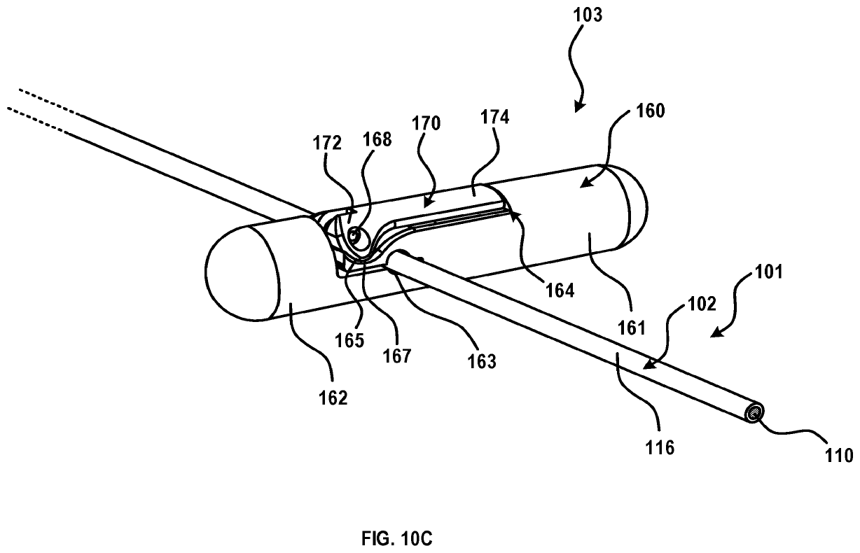

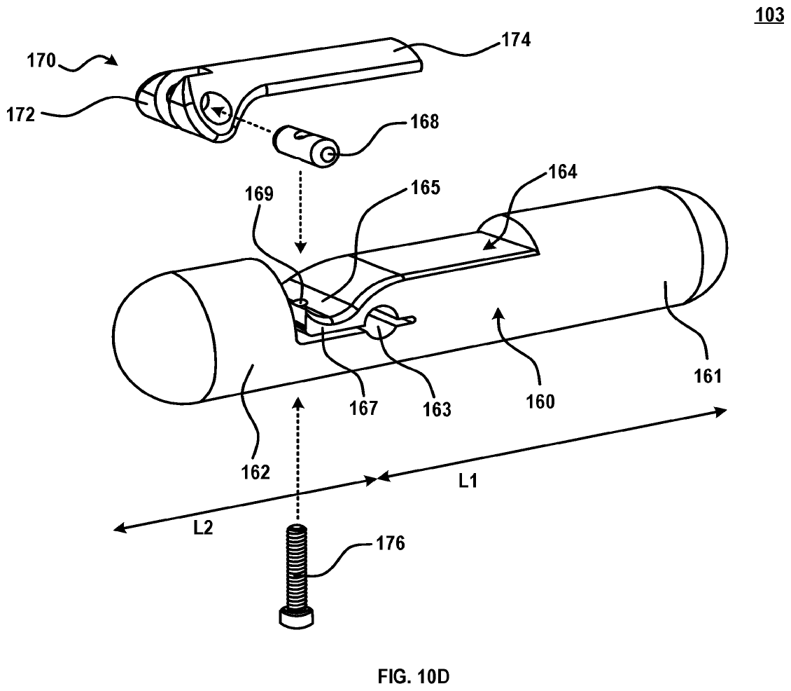

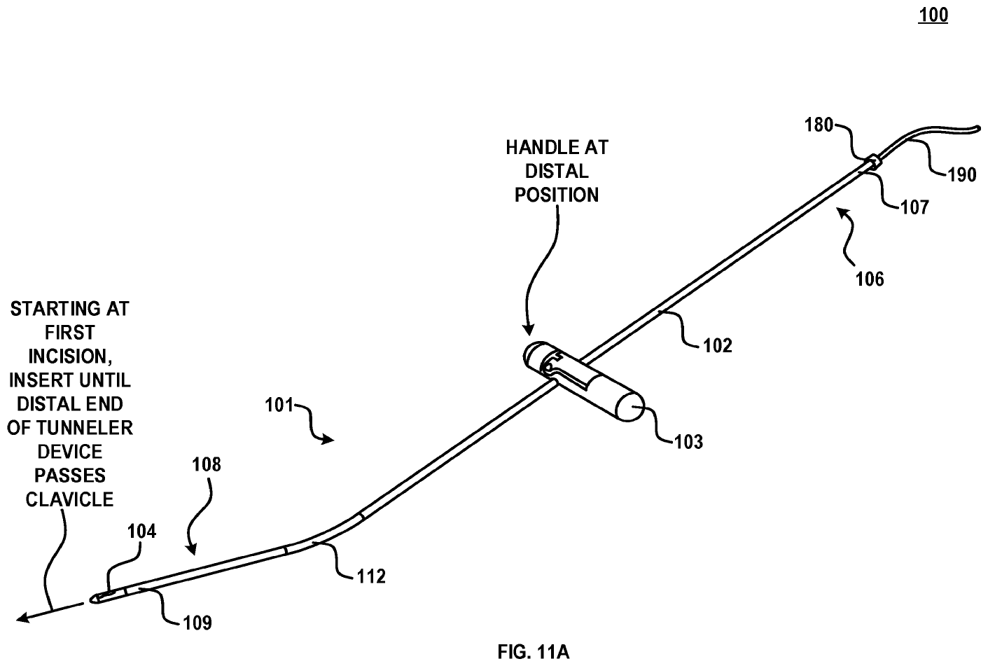

The handle 103 is shown in FIGS. 10A-10D and aids the practitioner in inserting the tunneler device 101 into the body. As shown, the handle 103 is operable for assuming a first open configuration (FIG. 10A) and a second closed configuration (FIG. 10B). The handle 103 is operable for displacement along the shaft 102 of the tunneler device 101 when in the first open configuration and can be fixed at a selected position along the shaft 102 when the handle 103 is in the second closed configuration. In particular, the handle 103 captures the shaft 102 in a clamping arrangement when in the second closed configuration and when mounted along an external surface 116 of the shaft 102 as shown in FIG. 10C. FIG. 10D provides an exploded view of the handle 103, including a handle body 160 having a first side 161 and a second side 162 divided by a conduit 163; the conduit 163 receives the shaft 102 of the tunneler device 101. The first side 161 defines a first length L1 and the second side 162 defines a second length L2; in a primary embodiment, the first length L1 of the first side 161 is substantially longer than the second length L2 of the second side 162 and the first side 161 and the second side 162 extend in a perpendicular direction relative to a direction of elongation of the shaft 102 of the tunneler device 101. This arrangement provides a “pistol-grip” configuration to improve a practitioner's grasp on the handle 103 for improved control when inserting the tunneler device 101 into the body.

As shown, the handle body 160 includes a recess 164 that receives an arm 170 for transitioning the handle 103 between the first open configuration and the second closed configuration. The arm 170 extends in a perpendicular direction from the handle body 160 when in the first open configuration and lies flat against the recess 164 when in the second closed configuration. The handle body 160 includes a biased tab 167 in association with the conduit 163 and the arm 170. When the arm 170 is in the first open configuration, the biased tab 167 is allowed to assume a resting configuration such that the conduit 163 “loosens” around the shaft 102 of the tunneler device 101, enabling a practitioner to adjust a position of the handle 103 along the shaft 102. When the arm 170 is in the second closed configuration, the biased tab 167 is compressed towards a center of the handle body 160 such that the conduit 163 is secure against the shaft 102 of the tunneler device 101 and a selected position of the handle 103 is fixed along the shaft 102. As shown, the arm 170 provides a bulbous portion 172 opposite from a distal arm portion 174; the recess 164 of the handle body 160 includes a valley portion 165 that receives the bulbous portion 172 of the arm 170. The bulbous portion 172 of the arm 170 seats within the valley portion 165 of the handle body 160 at a hinge 168 in association with a securing element 176.

The securing element 176 is configured for insertion through a securing aperture 169 associated with the valley portion 165 that extends through the handle body 160 to couple with the hinge 168, which in turn couples with the bulbous portion 172 of the arm 170. When the securing element 176 is completely tightened against the handle body 160, the biased tab 167 is compressed towards the center of the handle body 160 in the second closed configuration. Conversely, when the securing element 176 is loosened relative to the handle body 160, the biased tab 167 is allowed to relax away from the center of the handle body 160 in the first open configuration. As such, to transition the handle 103 into the first open configuration, a practitioner can loosen the securing element 176 to relax the biased tab 167 and loosen the conduit 163 around the shaft 102, which enables rotation of the arm 170 to the first open configuration shown in FIG. 10A. To transition the handle 103 into the second closed configuration shown in FIG. 10B, a practitioner can flatten the arm 170 against the recess 164 of the handle body 160 and tighten the securing element 176 against the biased tab 167, thereby capturing the shaft 102 within the conduit 163 such that the handle 103 is fixed at the selected position along the shaft 102 of the tunneler device 101.

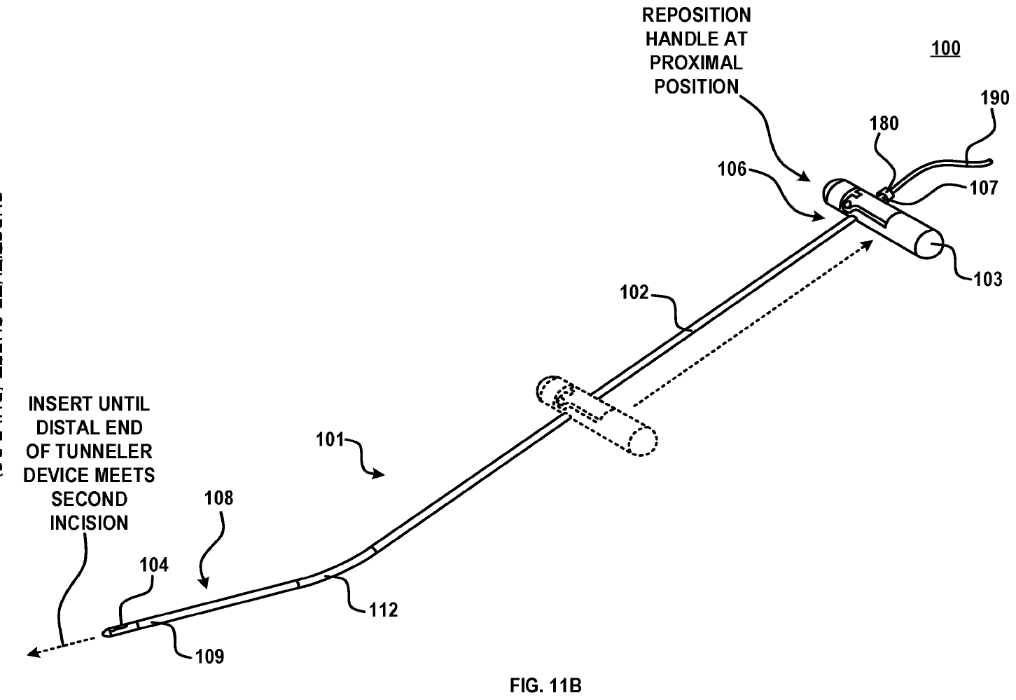

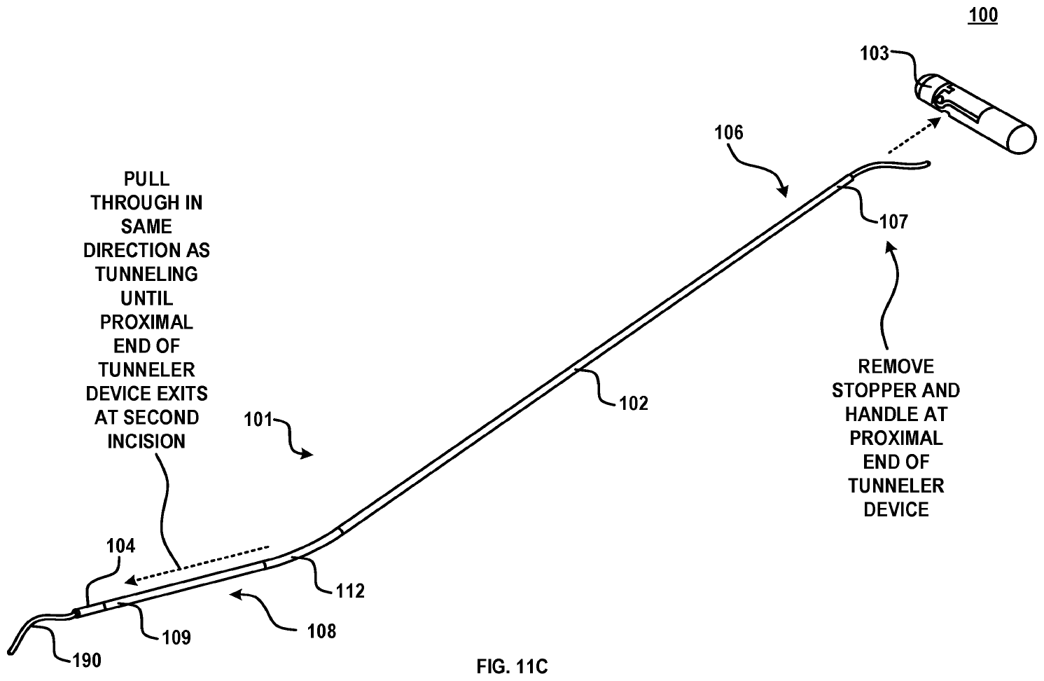

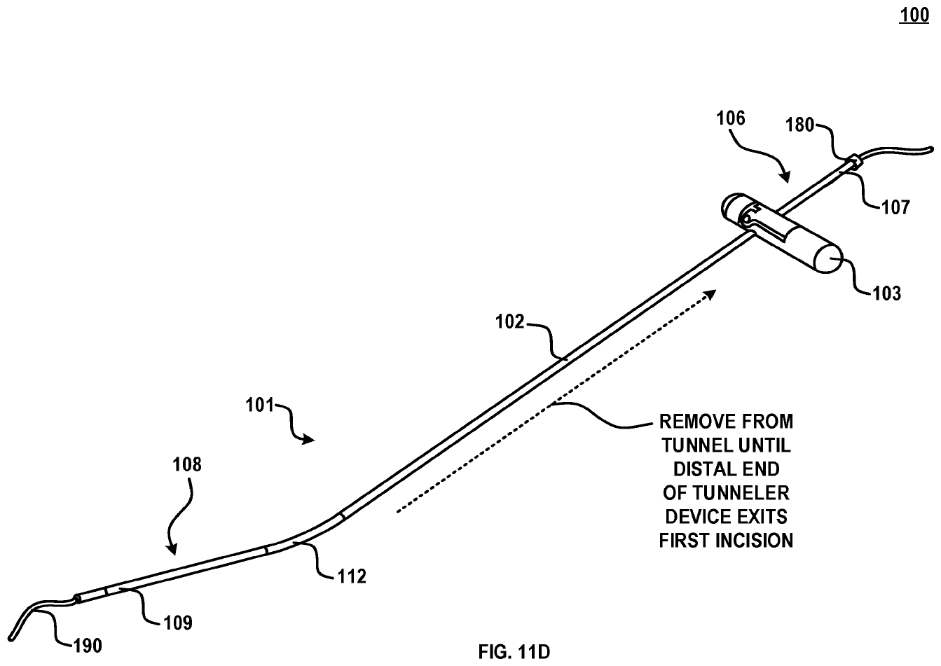

FIGS. 11A-11C show displacement of the handle 103 along the shaft 102 of the tunneler device 101 at different stages of insertion of the tunneler device 101 into the body. In particular, with reference to FIG. 11A, when inserting the tunneler device 101 into the body 1 (FIG. 3A), a practitioner inserts the distal portion 108 of the tunneler device 101 into the first incision 10 (FIG. 3A) until the distal end 109 passes the clavicle 40 (FIG. 3A) of the body 1. At this stage, it can be helpful to position the handle 103 at a distal position along the shaft 102 of the tunneler device 101 as shown in FIG. 11A. Referring now to FIG. 11B, the practitioner can re-position the handle 103 at a proximal position along the shaft 102 of the tunneler device 101 to aid in advancing the tunneler device 101 through the body 1 to form the tunnel 30 (FIG. 3A). The practitioner can re-position the handle 103 as many times as necessary to aid in advancing the tunneler device 101 through the body 1. Once tunneling is completed and the distal end 109 reaches the second incision 20 (FIG. 3A), then the tunneler device 101 can be removed from the tunnel 30 following decoupling of the medical tube 190 from the distal end 109 of the tunneler device 101. Two options are presented in FIGS. 11C and 11D; as shown in FIG. 11C, the stopper 180 can be removed from the proximal end 107 of the tunneler device 101, enabling subsequent removal of the handle 103 from the proximal portion 106 of the tunneler device 101. Following removal of the handle 103, the tunneler device 101 can be pulled through the tunnel 30 towards the second incision 20 in the same direction as tunneling until the proximal end 107 of the tunneler device 101 exits at the second incision 20. Conversely, as shown in FIG. 11D, the tunneler device 101 can be pulled through the tunnel 30 towards the first incision 10 in the opposite direction as tunneling until the distal end 109 of the tunneler device 101 exits at the first incision 10.

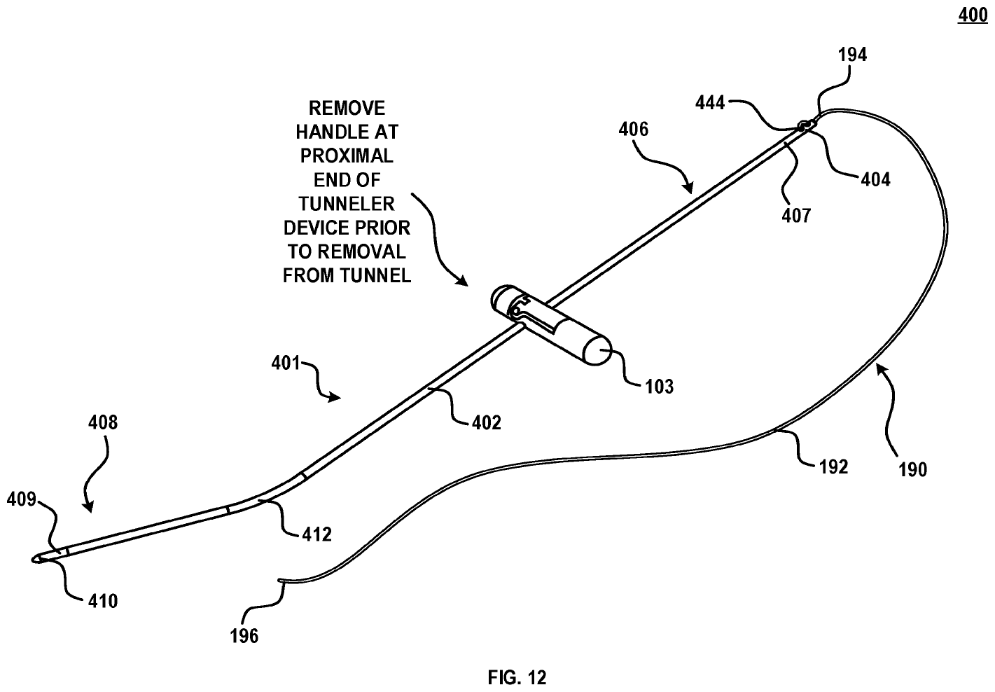

FIG. 12 shows an alternate embodiment of the system 400 that provides a tunneler device 401 having a shaft 402 defining a proximal portion 406 and a distal portion 408, where the shaft 402 is solid. As shown, the shaft 402 can similarly include a bend angle 412. In this embodiment, the distal portion 194 of the medical tube 190 can couple at a proximal end 407 of the shaft 402 rather than a distal end 409 of the shaft 402. The distal end 409 of the shaft 402 can include a tapered distal end 410 to tunnel through the body. The proximal end 407 of the shaft 402 can include an attachment element 404 having a receptacle 444 for capturing the distal portion 194 of the medical tube 190. As the tunneler device 401 advances through the body, the tunneler device 401 forms the tunnel 30 (FIG. 3A) between the first incision 10 and the second incision 20. In this embodiment, removal of the tunneler device 401 results in concurrent insertion of the medical tube 190 within the tunnel 30. In particular, the tunneler device 401 can be removed by pulling the tunneler device 401 through the second incision 20 similar to the method outlined in FIG. 11C with reference to the tunneler device 101. In this embodiment, when the proximal end 407 exits the second incision 20, the distal portion 194 of the medical tube 190 is positioned at the second incision 20 and the proximal portion 196 of the medical tube 190 is positioned at the first incision 10 such that the tube body 192 remains within the tunnel 30 as shown in FIG. 3B.

As shown, the tunneler device 401 can include the handle 103; the position of the handle 103 along the shaft 402 can be similarly adjusted according to the methods outlined with reference to FIGS. 10A-11C as the practitioner sees fit to aid in tunneling. Prior to removal of the tunneler device 401 from the tunnel 30, the handle 103 can be removed at the proximal end 407 of the tunneler device 401 similar to the method shown in FIG. 11C. Care should be taken to avoid damaging the medical tube 190 when removing the handle 103.

In some embodiments, the tunneler device 101 or 401 may be reusable. For a reusable embodiment, the tunneler device 101 or 401 can include a metallic material, such as stainless steel, substantially steel alloys, surgical grade steel, substantially titanium alloys, and substantially aluminum alloys. For a reusable embodiment, the tunneler device 101 or 401 may need to be sterilized via autoclave. In other embodiments, the tunneler device 101 or 401 can be disposable to prevent transmission of infection between patients. In addition, the tunneler device 101 or 401 may comprise a biocompatible material.

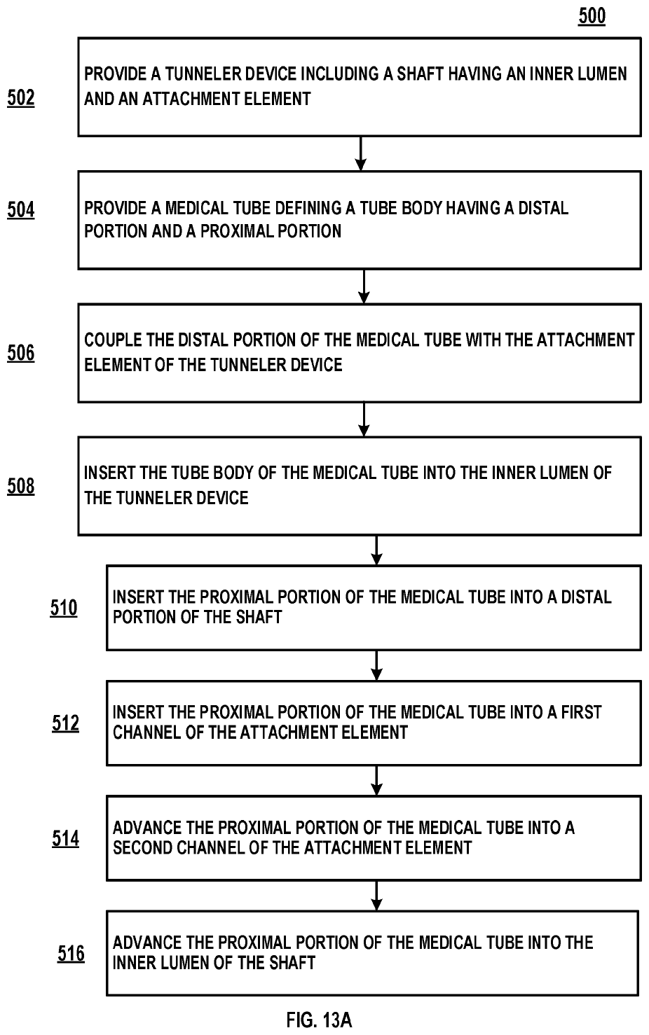

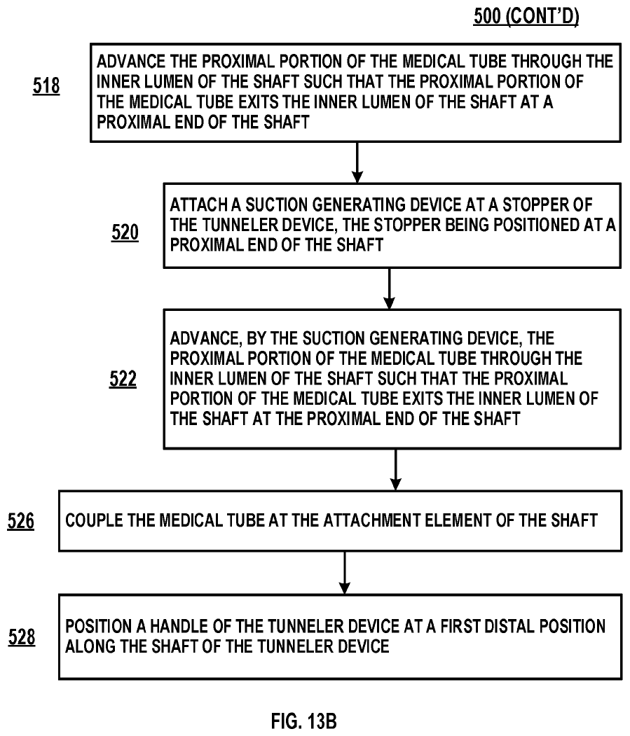

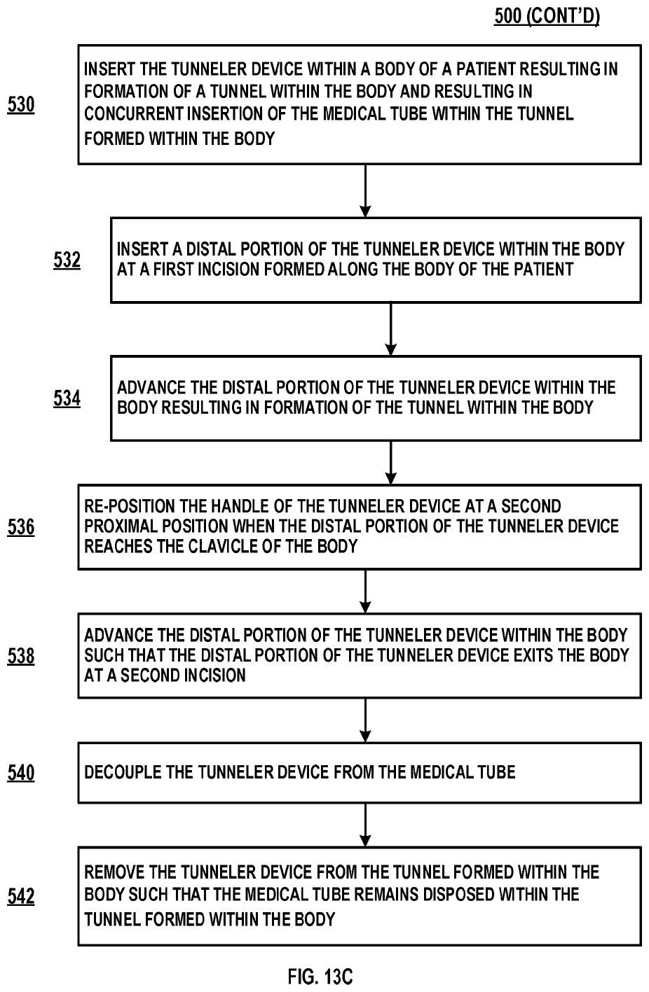

FIGS. 13A-13C show a method 500 for inserting a medical tube by the system 100 of FIGS. 1-11D. The method 500 starts at block 502, which includes providing a tunneler device including a shaft having an inner lumen and an attachment element. Block 504 includes providing a medical tube defining a tube body having a distal portion and a proximal portion. Block 506 includes coupling the distal portion of the medical tube with the attachment element of the tunneler device. Block 508 includes inserting the tube body of the medical tube into the inner lumen of the tunneler device.

Block 508 can be divided into several sub-steps, including blocks 510-518. Block 510 includes inserting the proximal portion of the medical tube into a distal portion of the shaft. Block 512 includes inserting the proximal portion of the medical tube into a first channel of the attachment element. Block 514 includes advancing the proximal portion of the medical tube into a second channel of the attachment element. Block 516 includes advancing the proximal portion of the medical tube into the inner lumen of the shaft. Block 518 includes advancing the proximal portion of the medical tube through the inner lumen of the shaft such that the proximal portion of the medical tube exits the inner lumen of the shaft at a proximal end of the shaft. In some embodiments, block 518 can be divided further into blocks 520 and 522; block 520 includes attaching a suction generating device at a stopper of the tunneler device, the stopper being positioned at a proximal end of the shaft and block 522 includes advancing, by the suction generating device, the proximal portion of the medical tube through the inner lumen of the shaft such that the proximal portion of the medical tube exits the inner lumen of the shaft at the proximal end of the shaft. In other embodiments where the advancement step of block 518 is performed manually without the aid of the suction generating device, blocks 520 and 522 can be omitted.

Block 526 includes coupling the medical tube at the attachment element of the shaft. Note that in some embodiments, this step can be performed prior to or following the advancement step in block 518. Block 528 includes positioning a handle of the tunneler device at a first distal position along the shaft of the tunneler device. Note that this step can be performed at the practitioner's discretion.

Block 530 includes inserting the tunneler device within a body of a patient resulting in formation of a tunnel within the body and resulting in concurrent insertion of the medical tube within the tunnel formed within the body. Block 530 can include sub-steps outlined in blocks 532 and 534; block 532 includes inserting a distal portion of the tunneler device within the body at a first incision formed along the body of the patient and block 534 includes advancing the distal portion of the tunneler device within the body resulting in formation of the tunnel within the body.

Block 536 can include re-positioning the handle of the tunneler device at a second proximal position when the distal portion of the tunneler device reaches the clavicle of the body. Note that this step can be performed and repeated at the practitioner's discretion as the tunneler device advances through the body similar to that of block 528.

Block 538 includes advancing the distal portion of the tunneler device within the body such that the distal portion of the tunneler device exits the body at a second incision. Block 540 includes decoupling the tunneler device from the medical tube. Finally, block 542 includes removing the tunneler device from the tunnel formed within the body such that the medical tube remains disposed within the tunnel formed within the body, which can be performed as shown in the examples of FIG. 11C or FIG. 11D.

Regarding the system 400 outlined above with reference to FIG. 12, aspects of blocks 502-506, and 526-542 of method 500 still apply. In this case, with respect to block 502, the shaft of the tunneler device of system 400 does not include an inner lumen; as such, the distal portion of the medical tube couples at the attachment element positioned at the proximal portion of the shaft. With respect to block 530, for the embodiment of FIG. 12, the medical tube is not concurrently inserted with formation of the tunnel but is instead concurrently inserted within the tunnel upon removal of the tunneler device from the tunnel. As such, block 540 that includes decoupling the tunneler device from the medical tube can be performed after the removal step in block 542.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A medical device, comprising:
a tunneler device including a shaft including an inner lumen, a proximal portion defining a proximal end, the proximal end being open and providing access to the inner lumen, a distal portion defining a distal end, and an attachment element positioned at the distal end of the shaft;
wherein the attachment element is configured to capture a distal portion of a medical tube;
wherein the tunneler device is configured for insertion within a body of a patient resulting in formation of a tunnel within the body; and
wherein insertion of the tunneler device within the body results in concurrent insertion of the medical tube within the body.

2. The medical device of claim 1, wherein the inner lumen is configured to receive the medical tube prior to insertion of the tunneler device within the body.

3. The medical device of claim 1, wherein the attachment element occludes the inner lumen at the distal end of the shaft.

4. The medical device of claim 1, wherein the shaft defines a bend angle along the distal portion of the shaft.

5. The medical device of claim 1, wherein the attachment element includes at least one receptacle that captures a distal portion of the medical tube in a friction-fit engagement.

6. The medical device of claim 1, wherein the attachment element defines a first channel that communicates with a second channel, the first channel being in communication with an exterior of the shaft and the second channel being in coaxial alignment with the inner lumen of the shaft.

7. The medical device of claim 1, wherein the attachment element defines a tapered distal portion.

8. The medical device of claim 1, wherein the attachment element is permanently fixed to the shaft.

9. The medical device of claim 1, wherein the attachment element defines a first cylindrical section configured for insertion within a distal portion of the shaft.

10. The medical device of claim 9, wherein the first cylindrical section includes a biased portion having a button along an outer surface of the biased portion, the biased portion being biased in a first radial direction away from a central axis of the attachment element and wherein the distal portion of the shaft includes an aperture configured to receive the button such that actuation of the button in a second radial direction towards the central axis of the attachment element enables decoupling of the attachment element from the distal portion of the shaft.

11. The medical device of claim 9, wherein a proximal portion of the first cylindrical section includes a barb that captures the distal portion of the medical tube in a friction-fit engagement.

12. A medical device, comprising:
a tunneler device including a shaft having an inner lumen and a handle slidably mounted along the shaft, the inner lumen being configured to receive a medical tube;
wherein the tunneler device is configured for insertion within a body of a patient resulting in formation of a tunnel within the body; and
wherein insertion of the tunneler device within the body results in concurrent insertion of the medical tube within the tunnel formed within the body.

13. The medical device of claim 12, wherein the handle is operable to assume a first open configuration and a second closed configuration.

14. The medical device of claim 13, wherein the handle is operable for displacement along the shaft when the handle is in the first open configuration.

15. The medical device of claim 13, wherein the handle is fixed at a selected position along the shaft when the handle is in the second closed configuration.

16. The medical device of claim 13, wherein the handle captures the shaft in a clamping arrangement when in the second closed configuration and when mounted along the shaft.

17. The medical device of claim 12, wherein the handle includes a first side and a second side and having a conduit therebetween, wherein the conduit aligns with a direction of elongation of the shaft when the handle is coupled along the shaft and wherein the first side and the second side extend in a perpendicular direction relative to the direction of elongation of the shaft.

18. The medical device of claim 17, wherein a first length of the first side is substantially greater than a second length of the second side.

19. The medical device of claim 12, wherein the handle is removably coupled along the shaft.

20. The medical device of claim 12, further comprising a stopper removably coupled at a proximal end of the shaft.

21. A system, comprising:
a tunneler device including a shaft including an inner lumen, a proximal portion defining a proximal end, the proximal end being open and providing access to the inner lumen, a distal portion defining a distal end and an attachment element positioned at the distal end, wherein the tunneler device is configured for insertion within a body of a patient resulting in formation of a tunnel within the body; and
a medical tube defining a tube body having a distal portion removably coupled to the attachment element of the shaft and configured to be disposed within the inner lumen of the tunneler device prior to insertion of the tunneler device within the body;
wherein insertion of the tunneler device within the body results in concurrent insertion of the medical tube within the body; and
wherein the tunneler device is configured for removal from the tunnel formed within the body such that the medical tube remains within the tunnel.

22. The system of claim 21, wherein the attachment element occludes the inner lumen at the distal end of the shaft.

23. The system of claim 21, wherein the shaft defines a bend angle along the distal portion of the shaft.

24. The system of claim 21, wherein the attachment element includes at least one receptacle that captures the distal portion of the medical tube in a friction-fit engagement.

25. The system of claim 21, wherein the attachment element includes a tapered distal portion.

26. The system of claim 21, further comprising a handle slidably mounted along an external surface of the shaft.

27. The system of claim 26, wherein the handle is removably coupled along the shaft.

28. The system of claim 21, further comprising a stopper positioned at the proximal end of the shaft and configured to couple with a suction generating device, wherein the stopper includes a stopper aperture that enables access to the inner lumen at the proximal end of the shaft.

29. A method, comprising:
providing a tunneler device including a shaft having an inner lumen and an attachment element;
providing a medical tube defining a tube body having a distal portion;
coupling the distal portion of the medical tube with the attachment element of the tunneler device;
inserting the tube body of the medical tube into the inner lumen of the tunneler device;
inserting the tunneler device within a body of a patient resulting in formation of a tunnel within the body and resulting in concurrent insertion of the medical tube within the tunnel formed within the body; and
removing the tunneler device from the tunnel formed within the body such that the medical tube remains disposed within the tunnel formed within the body.

30. The method of claim 29, further comprising:
inserting a proximal portion of the medical tube into the inner lumen of the shaft; and
advancing the proximal portion of the medical tube through the inner lumen of the shaft such that the proximal portion of the medical tube exits the inner lumen of the shaft at a proximal end of the shaft.

31. The method of claim 30, further comprising:
inserting the proximal portion of the medical tube into a first channel of the attachment element;
advancing the proximal portion of the medical tube into a second channel of the attachment element; and
advancing the proximal portion of the medical tube into the inner lumen of the shaft.

32. The method of claim 30, further comprising:
attaching a suction generating device at a stopper of the tunneler device, the stopper being positioned at a proximal end of the shaft; and
advancing, by the suction generating device, the proximal portion of the medical tube through the inner lumen of the shaft such that the proximal portion of the medical tube exits the inner lumen of the shaft at the proximal end of the shaft.

33. The method of claim 30, further comprising:
coupling the distal portion of the medical tube with one or more receptacles of the attachment element following insertion of the medical tube within the inner lumen.

34. The method of claim 30, further comprising:
coupling the distal portion of the medical tube with one or more receptacles of the attachment element prior to insertion of the medical tube within the inner lumen;
inserting the proximal portion of the medical tube into a distal portion of the shaft;
advancing the proximal portion of the medical tube through the inner lumen of the shaft such that the proximal portion of the medical tube exits the inner lumen of the shaft at a proximal end of the shaft; and
coupling the attachment element at the distal portion of the shaft.

35. The method of claim 29, further comprising:
positioning a handle of the tunneler device at a first distal position along the shaft of the tunneler device.

36. The method of claim 35, further comprising:
positioning the handle of the tunneler device at a second proximal position along the shaft of the tunneler device following insertion of the tunneler device within the body.

37. The method of claim 29, further comprising:
inserting a distal portion of the tunneler device within the body at a first incision formed along the body of the patient;
advancing the distal portion of the tunneler device within the body resulting in formation of the tunnel within the body; and
advancing the distal portion of the tunneler device within the body such that the distal portion of the tunneler device exits the body at a second incision.

38. The method of claim 37, wherein the first incision is positioned superior to a clavicle of the body and wherein the second incision is positioned inferior to the clavicle of the body.

39. The method of claim 38, further comprising:
positioning a handle of the tunneler device at a second proximal position is performed when a distal portion of the tunneler device reaches the clavicle of the body.

40. The method of claim 29, further comprising:
decoupling the tunneler device from the medical tube prior to removal of the tunneler device from the body.

41. A system, comprising:
a tunneler device including a shaft having an attachment element at a proximal end of the shaft; and
a medical tube defining a tube body having a distal portion removably coupled at the attachment element of the shaft;
wherein the tunneler device is configured for insertion within a body of a patient resulting in formation of a tunnel within the body;
wherein removal of the tunneler device within the body results in concurrent insertion of the medical tube within the tunnel formed within the body.

42. The system of claim 41, wherein the attachment element includes at least one receptacle that captures the distal portion of the medical tube.

43. The system of claim 42, wherein the at least one receptacle captures the distal portion of the medical tube in a friction-fit engagement.

44. The system of claim 41, further comprising a handle slidably mounted along an external surface of the shaft.

45. The system of claim 44, wherein the handle is removably coupled along the shaft.

46. The system of claim 41, wherein the shaft includes a distal portion defining a distal end, wherein the distal end of the shaft includes a tapered distal portion.
